(19) United States
(12) Patent Application Publication
(10) Pub. No.: US 2008/0249303 A1
(43) Pub. Date: Oct. 9, 2008

(54) METHODS OF SEPARATION AND DETECTION OF BAZEDOXIFENE ACETATE IN PHARMACEUTICAL COMPOSITIONS

(75) Inventors: Wei TONG, Suffern, NY (US); Carl E. LONGFELLOW, Warwick, NY (US)

Correspondence Address: WilmerHale/Wyeth, 60 STATE STREET, BOSTON, MA, 02109, US

(73) Assignee: WYETH, Madison, NJ (US)

(21) Appl. No.: 12/057,465

(22) Filed: Mar. 28, 2008

Related U.S. Application Data
(60) Provisional application No. 60/909,113, filed on Mar. 30, 2007.

(51) Int. Cl. C07D 401/12; G01N 23/20

(52) U.S. Cl. 540/602; 378/71

(57) ABSTRACT

Methods are disclosed for separating and detecting bazedoxifene acetate from pharmaceutical compositions containing a mixture of bazedoxifene acetate and one or more other components that produce X-Ray diffraction patterns having interfering peaks at or near the characteristic peaks for bazedoxifene acetate.

### CROSS REFERENCE TO RELATED APPLICATIONS

This application claims the benefit of priority under 35 U.S.C. §119(e) to U.S. Provisional Patent Application No. 60/909,113 filed on Mar. 30, 2007, which is hereby incorporated by reference in its entirety.

### FIELD

The disclosure relates to methods of separating and detecting bazedoxifene acetate from pharmaceutical compositions containing a mixture of bazedoxifene acetate and one or more other components. Methods of separating and detecting crystalline bazedoxifene acetate Form A and/or Form B in pharmaceutical compositions are disclosed.

### BACKGROUND

Bazedoxifene is an estrogenic agent which is useful in treating a variety of conditions, disorders or diseases, particularly those associated with menopause. Bazedoxifene salts, particularly, bazedoxifene acetate is used in pharmaceutical formulations. Bazedoxifene acetate (1-[4-(2-azepan-1-yl-ethoxy)-benzyl]-2-(4-hydroxy-phenyl)-3-methyl-1H-indol-5-ol acetic acid) has the chemical formula shown below.

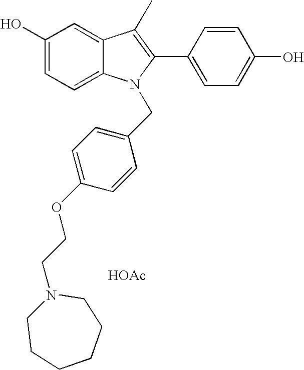

Bazedoxifene belongs to the class of drugs typically referred to as selective estrogen receptor modulators (SERMs). Consistent with its classification, bazedoxifene demonstrates affinity for estrogen receptors (ER) but shows tissue selective estrogenic effects. Bazedoxifene acetate has demonstrated estrogenic activity on bone and cardiovascular lipid parameters and antiestrogenic activity on uterine and mammary tissue and thus has the potential for treating a number of different disease or disease-like states involving the estrogen receptor.

U.S. Pat. Nos. 5,998,402 and 6,479,535 report the preparation of bazedoxifene acetate. The synthetic preparation of bazedoxifene acetate has also appeared in the general literature. See, for example, Miller et al., J. Med. Chem., 2001, 44, 1654-1657. Description of the drug's biological activity has appeared in the general literature as well (e.g. Miller, et al. Drugs of the Future, 2002, 27(2), 117-121). In addition, pharmaceutical compositions containing bazedoxifene acetate is disclosed in WO 02/03987. Further, co-administration of bazedoxifene acetate and conjugated estrogens is disclosed in U.S. Pat. No. 6,479,535 and US 2007/0003623, as well as in commonly assigned and co-pending patent application Ser. No. 11/946,586, filed on Nov. 28, 2007, and U.S. Ser. No. 12/013,109, filed on Jan. 11, 2008.

The crystalline polymorph form of a particular drug often affects the drug's ease of preparation, stability, solubility, storage stability, ease of formulation and in vivo pharmacology. Polymorphic forms occur where the same composition of matter crystallizes in a different lattice arrangement resulting in different thermodynamic properties and stabilities specific to the particular polymorph form. In cases where two or more polymorphs can be produced, it may be desirable to have a method to make each polymorph in pure form.

Polymorphic Form A of bazedoxifene acetate is disclosed in US 2005/0227965 while polymorphic Form B of bazedoxifene acetate is disclosed in US 2005/0250762. Methods of preparing polymorphic Form A of bazedoxifene acetate are also disclosed in commonly assigned and co-pending patent application Ser. Nos. 61/027,607 and 61/027,634, filed on Feb. 11, 2008. Form A has higher solubility in both aqueous and organic solvent systems than Form B. This can be advantageous in formulations or doses where the solubility of the particular composition is of concern. For example, higher solubility can influence bioavailability, which can affect biological absorption and distribution of the drug and can facilitate formulation in liquid carriers. However, Form A is the kinetic (or meta-stable) polymorph while Form B is the thermodynamically more stable polymorph. Form A can easily convert to Form B upon contact with a solvent or solvent mixture (e.g., ethyl acetate and ethanol), which presents a challenge to the preparation of pure Form A that is substantially free of Form B. Further, under various conditions, and over time, Form A can convert to Form B.

Accordingly, it is useful to detect levels of Form A and Form B in pharmaceutical compositions, for example, to detect the presence of Form B in pharmaceutical compositions of Form A, to monitor the levels, if any, of conversion from Form A to Form B under various conditions and/or over time.

### SUMMARY

It has been discovered that some excipients commonly found in pharmaceutical compositions of bazedoxifene acetate, for example, lactose and sucrose, interfere with bazedoxifene acetate polymorphs in terms of X-ray diffraction (XRD) pattern. If bazedoxifene acetate tablets are tested using previous methods, i.e., without performing the extraction methods described herein, the detection limits for Form B are estimated at about 10% by weight relative to total bazedoxifene acetate for tablets containing 45.1 mg of bazedoxifene acetate (40 mg of bazedoxifene as free base) and 20% by weight relative to total bazedoxifene acetate for tablets containing 22.6mg of bazedoxifene acetate (20 mg of bazedoxifene as free base), based on bazedoxifene acetate weight percentage in the tablets and instrument noise levels. If interfering excipients, such as lactose and sucrose, can be removed while crystalline bazedoxifene acetate Form A and Form B are retained, Form B can, be detected with greater sensitivity.

Disclosed herein are improved methods of separating and detecting crystalline bazedoxifene acetate Form A and/or Form B in pharmaceutical compositions.

In one aspect, methods are provided for separating a pharmaceutical composition comprising bazedoxifene acetate and one or more components that produce X-Ray diffraction patterns having one or more interfering peaks at or near the characteristic peak or peaks for bazedoxifene acetate. The method includes:

(a) contacting the pharmaceutical composition with an extraction medium to produce a suspension, wherein bazedoxifene acetate is substantially insoluble in the extraction medium and wherein the one or more components having one or more interfering peaks are substantially soluble in the extraction medium;

(b) filtering the suspension to produce a filtrate and a filtrand, wherein the one or more components are substantially contained in the filtrate; and

(c) drying the filtrand to produce a composition substantially free of the one or more components that produce X-Ray diffraction patterns having one or more interfering peaks.

In certain embodiments, the bazedoxifene acetate is substantially contained in the filtrand. In certain embodiments, the method further comprises washing the filtrand.

In certain embodiments, the method further comprises forming the composition obtained in step (c) mentioned above into a tablet or pellet for X-Ray diffraction measurement.

In certain embodiments, the bazedoxifene acetate is bazedoxifene acetate Form A and/or bazedoxifene acetate Form B. In certain embodiments, the characteristic peak for bazedoxifene acetate Form A is at about 12.8±0.2° in 2theta angular degree by Cu radiation, and the characteristic peaks for bazedoxifene acetate Form B are at about 12.0±0.2° and 13.3±0.2° in 2theta angular degree by Cu radiation.

In certain embodiments, the one or more components that produce X-Ray diffraction patterns having interfering peaks include, without limitation, pharmaceutically acceptable diluents, fillers, excipients, binding agents, lubricants, disintegrants, suspending or stabilizing agents, and mixtures thereof. In certain embodiments, the one or more components that produce X-Ray diffraction patterns having interfering peaks include lactose, sucrose, or mixtures thereof. In some embodiments, the one or more components that produce X-Ray diffraction patterns having interfering peaks include lactose.

In certain embodiments, the interfering peaks are at from about 11.6±0.2° to about 13.7±0.2° in 2theta angular degree by Cu radiation.

In certain embodiments, the extraction medium is a solution including one or more acetate salts. In certain embodiments, the solution includes ammonium acetate, sodium acetate, potassium acetate, magnesium acetate, calcium acetate, or mixtures thereof. In certain embodiments, the solution comprises ammonium acetate, sodium acetate, or mixtures thereof. In some cases, the solution comprises sodium acetate.

In certain embodiments, the solution has a concentration of about 0.05 M to about 1 M with respect to acetate. In certain embodiments, the solution has a concentration of about 0.25 M to about 0.75 M with respect to acetate. In some embodiments, the solution has a concentration of about 0.45 M to about 0.55 M with respect to acetate.

In certain embodiments, the solution has a pH range from about 5 to about 10. In certain embodiments, the solution has a pH range from about 6 to about 9.2. In some embodiments, the solution has a pH range from about 6.2 to about 8.5.

In certain embodiments, the pharmaceutical composition is provided as at least one unit dosage form. Examples of unit dosage forms include, without limitation, tablet, capsule, gel cap, buccal form, troche, and lozenge. In certain embodiments, the unit dosage form is tablet.

In certain embodiments, the method further comprises removing any coating from the pharmaceutical composition prior to contacting the composition with the extraction medium.

In certain embodiments, the amount of the extraction medium used during the contacting of the pharmaceutical composition with the extraction medium to produce the suspension is from about 0.2 ml per dosage (e.g., tablet) unit to about 10 ml per dosage unit. In certain embodiments, the pharmaceutical composition is contacted with the extraction medium for about 1 to about 120 minutes. In some embodiments, the pharmaceutical composition is contacted with the extraction medium for about 5 to about 30 minutes. In some embodiments, the pharmaceutical composition is contacted with the extraction medium for about 5 to about 15 minutes.

In another aspect, methods are provided for separating a pharmaceutical composition comprising bazedoxifene acetate Form A and/or Form B and one or more components that produce X-Ray diffraction patterns having one or more interfering peaks at or near the characteristic peak or peaks for bazedoxifene acetate Form A and/or Form B. The method includes:

(a) contacting the pharmaceutical composition with a solution comprising at least one acetate salt to produce a suspension, wherein bazedoxifene acetate Form A and/or Form B is substantially insoluble in the solution and wherein the one or more components are substantially soluble in the solution;

(b) filtering the suspension to produce a filtrate and a filtrand, wherein the one or more components are substantially contained in the filtrate; and (c) washing and drying the filtrand to produce a composition substantially free of the one or more components that produce X-Ray diffraction patterns having one or more interfering peaks.

In certain embodiments, the pharmaceutical composition is provided as tablet form, and the method further includes removing any coating from the tablet prior to contacting the tablet with the solution.

In certain embodiments, the one or more components that produce X-Ray diffraction patterns having interfering peaks include, without limitation, lactose, sucrose, and mixtures thereof.

In certain embodiments, the characteristic peak for bazedoxifene acetate Form A is at about 12.8±0.2° in 2theta angular degree by Cu radiation. In certain embodiments, the characteristic peaks for bazedoxifene acetate Form B are at about 12.0±0.2° and about 13.3±0.2° in 2theta angular degree by Cu radiation. In some embodiments, the interfering peaks are at from about 11.6±0.2° to about 13.7±0.2° in 2theta angular degree by Cu radiation.

In yet another aspect, a method is provided for detecting bazedoxifene acetate Form A and/or Form B in a pharmaceutical composition comprising bazedoxifene acetate Form A and/or Form B and one or more components (e.g., lactose) that produce X-Ray diffraction patterns having one or more interfering peaks at or near the characteristic peak or peaks for bazedoxifene acetate Form A and/or Form B. The method includes:

(a) producing a composition containing bazedoxifene acetate Form A and/or Form B by a method as described hereinabove, wherein the composition is substantially free of the components (e.g., lactose) that produce X-Ray diffraction patterns having one or more interfering peaks;

(b) forming the composition containing bazedoxifene acetate Form A and/or Form B into a tablet or pellet for X-Ray diffraction measurement; and

(c) analyzing the tablet or pellet using X-Ray diffraction.

In a further aspect, a method is provided for separating a pharmaceutical composition comprising bazedoxifene acetate and one or more components that produce X-Ray diffraction patterns having one or more interfering peaks at or near the characteristic peak or peaks for bazedoxifene acetate. The method includes:

(a) contacting the pharmaceutical composition with an extraction medium to produce a suspension, wherein bazedoxifene acetate is substantially insoluble in the extraction medium and wherein the one or more components are substantially soluble in the extraction medium;

(b) centrifuging the suspension to produce a solid and a supernatant solution, wherein the one or more components are substantially contained in the supernatant solution; and

(c) collecting and drying the solid to produce a composition substantially free of the one or more components that produce X-Ray diffraction patterns having one or more interfering peaks.

In certain embodiments, the pharmaceutical composition further includes conjugated estrogens. In certain embodiments, the pharmaceutical composition includes a core containing conjugated estrogens and an outer layer containing bazedoxifene acetate. In certain embodiments, the contacting of the pharmaceutical composition with an extraction medium in step (a) is stopped when a filler coating between the conjugated estrogen core and the bazedoxifene outer layer is exposed.

In certain embodiments, the bazedoxifene acetate is substantially contained in the solid provided by the centrifugation. In some embodiments, the method further comprises removing the supernatant solution produced in step (b). In some instances, the supernatant solution can be removed by decanting or through pipette. In some embodiments, the method further includes washing the solid produced in step (b).

In certain embodiments, the solid is collected by filtration. In certain embodiments, the method further comprises forming the composition obtained in step (c) into powder or a tablet or pellet for X-Ray diffraction measurement. In some embodiments, the method further includes analyzing the composition obtained in step (c) or the powder, tablet or pellet prepared from the composition using X-Ray diffraction.

In certain embodiments, the bazedoxifene acetate is bazedoxifene acetate Form A and/or bazedoxifene acetate Form B.

In certain embodiments, the characteristic peak for bazedoxifene acetate Form A is at about 12.8±0.2° in 2theta angular degree by Cu radiation, and the characteristic peaks for bazedoxifene acetate Form B are at about 12.0±0.2° and about 13.3±0.2° in 2theta angular degree by Cu radiation.

In certain embodiments, the one or more components that produce X-Ray diffraction patterns having interfering peaks include, without limitation, pharmaceutically acceptable diluents, fillers, excipients, binding agents, lubricants, disintegrants, suspending or stabilizing agents, and mixtures thereof. In certain embodiments, the one or more components that produce X-Ray diffraction patterns having interfering peaks include, without limitation, lactose, sucrose, or mixtures thereof. In some embodiments, the one or more components that produce X-Ray diffraction patterns having interfering peaks include, without limitation, sucrose.

In certain embodiments, the interfering peaks are at from about 11.9±0.2° to about 13.3±0.2° in 2theta angular degree by Cu radiation.

In certain embodiments, the extraction medium is a solution comprising one or more acetate salts. In certain embodiments, the solution includes ammonium acetate, sodium acetate, potassium acetate, magnesium acetate, calcium acetate, or mixtures thereof. In certain embodiments, the solution includes ammonium acetate, sodium acetate, or mixtures thereof.

In certain embodiments, the solution has a concentration of about 0.05 M to about 1 M with respect to acetate. In certain embodiments, the solution has a concentration of about 0.1 M to about 0.75 M with respect to acetate. In some embodiments, the solution has a concentration of about 0.1 M to about 0.3 M with respect to acetate.

In certain embodiments, the solution has a pH between about 5 and about 10. In certain embodiments, the solution has a pH between about 6 and about 9.2. In some embodiments, solution has a pH between about 6.2 and about 8.5.

In certain embodiments, the pharmaceutical composition is provided as at least one unit dosage form. Non-limiting examples of the unit dosage form include tablet, capsule, gel cap, buccal form, troche, and lozenge. In certain embodiments, the pharmaceutical composition is provided as a tablet. In some embodiments, the tablet form includes a core and an outer layer. In certain embodiments, the core contains conjugated estrogens and the outer layer contains bazedoxifene acetate.

In certain embodiments, the method further includes removing any coating from the pharmaceutical composition prior to contacting the composition with the extraction medium. In certain embodiments, the amount of the extraction medium used during the contacting of the pharmaceutical composition with the extraction medium to produce the suspension is from about 0.2 ml per dosage unit to about 10 ml per dosage unit (e.g., 10 mg, 20 mg or 40 mg bazidoxifene tablet, optionally containing conjugated estrogens). In some embodiments, the pharmaceutical composition is contacted with the extraction medium for about 1 to about 120 minutes. In some embodiments, the pharmaceutical composition is contacted with the extraction medium for about 1 to about 30 minutes. In some embodiments, the pharmaceutical composition is contacted with the extraction medium for about 1 to about 5 minutes. In some embodiments, the pharmaceutical composition is contacted with the extraction medium for about 2 minutes.

In another aspect, a method is provided for separating a pharmaceutical composition comprising bazedoxifene acetate Form A and/or Form B and one or more components that produce X-Ray diffraction patterns having one or more interfering peaks at or near the characteristic peak or peaks for bazedoxifene acetate Form A and/or Form B. The method includes:

(a) contacting the pharmaceutical composition with a solution comprising at least one acetate salt to produce a suspension, wherein bazedoxifene acetate Form A and/or Form B is substantially insoluble in the solution and wherein the one or more components are substantially soluble in the solution;

(b) centrifuging the suspension to produce a solid and a supernatant solution, wherein the one or more components are substantially contained in the supernatant solution; and

(c) collecting and drying the solid to produce a composition substantially free of the one or more components that produce X-Ray diffraction patterns having one or more interfering peaks.

In certain embodiments of, the pharmaceutical composition is provided as a tablet. In certain embodiments, the tablet includes a core and an outer layer. In some instances, the core contains conjugated estrogens and the outer layer contains bazedoxifene acetate Form A and/or Form B. In certain embodiments, the method further includes removing any coating from the tablet prior to contacting the tablet with the solution.

In certain embodiments, the one or more components that produce X-Ray diffraction patterns having interfering peaks include lactose, sucrose, or mixtures thereof. In certain embodiments, the one or more components that produce X-Ray diffraction patterns having interfering peaks include sucrose.

In certain embodiments, the characteristic peak for bazedoxifene acetate Form A is at about 12.8±0.2° in 2theta angular degree by Cu radiation. In certain embodiments, the characteristic peaks for bazedoxifene acetate Form B are at about 12.0±0.2° and 13.3±0.2° in 2theta angular degree by Cu radiation. In some embodiments, the interfering peaks are at from about 11.9±0.2° to about 13.3±0.2° in 2theta angular degree by Cu radiation.

In a further aspect, a method is provided for detecting bazedoxifene acetate Form A and/or Form B in a pharmaceutical composition comprising bazedoxifene acetate Form A and/or Form B and one or more components (e.g., sucrose) that produce X-Ray diffraction patterns having one or more interfering peaks at or near the characteristic peak or peaks for bazedoxifene acetate Form A and/or Form B. The method includes:

(a) producing a composition containing bazedoxifene acetate Form A and/or Form B by a method as described herein above, wherein the composition is substantially free of the one or more components (e.g., sucrose) that produce X-Ray diffraction patterns having one or more interfering peaks;

(b) forming the composition containing bazedoxifene acetate Form A and/or Form B into a tablet or pellet for X-Ray diffraction measurement; and

(c) analyzing the tablet or pellet using X-Ray diffraction.

### BRIEF DESCRIPTION OF THE DRAWINGS

FIG. 1 shows the X-Ray diffraction (XRD) patterns of bazedoxifene acetate Forms A and B, and lactose.

FIG. 2 shows the XRD patterns of bazedoxifene acetate Forms A and B, and sucrose.

### DETAILED DESCRIPTION

One aspect provides methods of separating a pharmaceutical composition comprising bazedoxifene acetate and one or more components that produce X-Ray diffraction patterns having one or more interfering peaks at or near the characteristic peak or peaks for bazedoxifene acetate. The method includes: (a) contacting the pharmaceutical composition with an extraction medium to produce a suspension, in which bazedoxifene acetate is substantially insoluble in the extraction medium and in which the one or more components are substantially soluble in the extraction medium; (b) filtering the suspension to produce a filtrate and a filtrand, in which the one or more components are substantially contained in the filtrate; and (c) drying the filtrand to produce a composition substantially free of the one or more components that produce X-Ray diffraction patterns having one or more interfering peaks.

In certain embodiments, the bazedoxifene acetate is substantially contained in the filtrand. In certain embodiments, the method further includes washing the filtrand.

In certain embodiments, the method further includes forming the composition obtained in step (c) mentioned above into powder or tablet or pellet for X-Ray diffraction measurement.

In certain embodiments, the bazedoxifene acetate is bazedoxifene acetate Form A and/or bazedoxifene acetate Form B. In some cases, the bazedoxifene acetate is a mixture of bazedoxifene acetate Form A and bazedoxifene acetate Form B. In certain embodiments, the characteristic peak for bazedoxifene acetate Form A is at about 12.8±0.2° in 2theta angular degree by Cu radiation, and the characteristic peaks for bazedoxifene acetate Form B are at about 12.0±0.2° and about 13.3±0.2° in 2theta angular degree by Cu radiation.

In certain embodiments, the one or more components (“interfering components”) that produce X-Ray diffraction patterns having interfering peaks include, without limitation, pharmaceutically acceptable diluents, fillers, excipients, binding agents, lubricants, disintegrants, suspending or stabilizing agents, and mixtures thereof. In some embodiments, the one or more components that produce X-Ray diffraction patterns having interfering peaks include, but are not limited to, one or more of magnesium stearate, stearic acid, talc, sodium lauryl sulfate, microcrystalline cellulose, ascorbic acid, sodium starch glycolate, pregelled starch, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, dry starches and powdered sugar. In certain embodiments, the one or more components that produce X-Ray diffraction patterns having interfering peaks include, without limitation, lactose, sucrose, or mixtures thereof. In some embodiments, the one or more components that produce X-Ray diffraction patterns having interfering peaks include, without limitation, lactose. In certain embodiments, the interfering peaks are at from about 11.6±0.2° to about 13.7±0.2° in 2theta angular degree by Cu radiation.

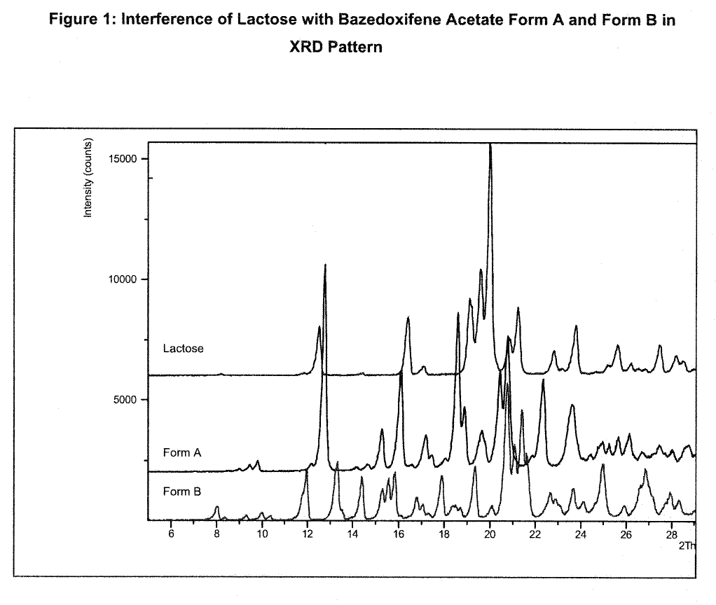

FIG. 1 illustrates the interference with the characteristic peaks of bazedoxifene acetate Forms A and B caused by lactose. In some embodiments, the peak of lactose at about 12.5±0.2° interferes with the characteristic peak of bazedoxifene acetate Form A at about 12.8±0.2° and the characteristic peaks of bazedoxifene acetate Form B at about 12.0±0.2°. In certain embodiments, the interfering peaks are at from about 11.9±0.20° to about 13.3±0.2°.

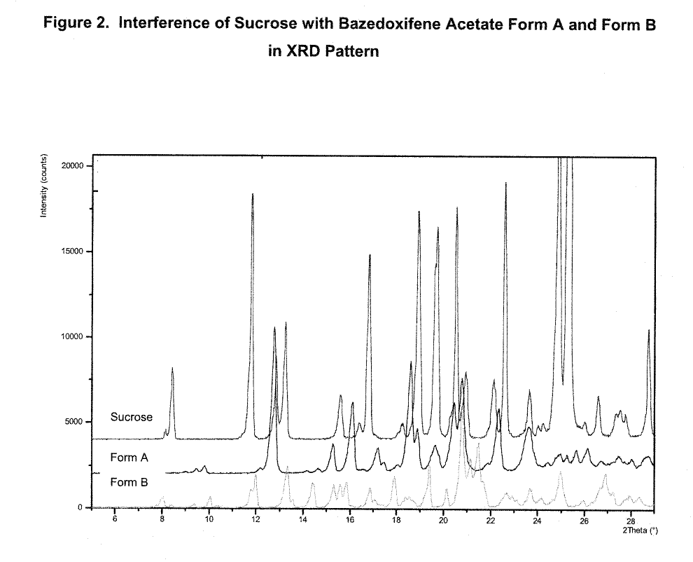

FIG. 2 illustrates the interference with the characteristic peaks of bazedoxifene acetate Forms A and B caused by sucrose. In some embodiments, the peak of sucrose at about 12.9±0.2° interferes with the characteristic peak of bazedoxifene acetate Form A at about 12.8±0.2°. In some other embodiments, the peak of sucrose at about 11.9±0.2° and 13.3±0.2° interferes with the characteristic peak of bazedoxifene acetate Form B at about 12.0±0.2° and 13.3±0.2° respectively.

In some embodiments of the methods described herein, bazedoxifene acetate is substantially insoluble in the extraction medium and the components that produce XRD patterns having interfering peaks at or near the characteristic peaks of bazedoxifene acetate are substantially soluble in the extraction medium.

The term “substantially insoluble” as used herein means “sparingly soluble,” “slightly soluble,” “very slightly soluble,” or “practically insoluble, or insoluble” as described in USP 25, The United States Pharmacopeia, page 2363 (2002). In certain embodiments, the term “substantially insoluble” with respect to bazedoxifene acetate means that less than about 1 mg of bazedoxifene acetate can dissolve in one mL of extraction solution, for example, less than about 0.5 mg of bazedoxifene acetate can dissolve in one mL of extraction solution, less than about 0.1 mg of bazedoxifene acetate can dissolve in one mL of extraction solution, less than about 0.05 mg of bazedoxifene acetate can dissolve in one mL of extraction solution, less than about 0.01 mg of bazedoxifene acetate can dissolve in one mL of extraction solution, or less than about 0.005 mg of bazedoxifene acetate can dissolve in one mL of extraction solution.

The term “substantially soluble” as used herein means “very soluble,” “freely soluble,” or “soluble” as described in USP 25, The United States Pharmacopeia, page 2363 (2002). In certain embodiments, the term “substantially soluble” as used herein with respect to an interfering component means that greater than about 20 mg of the interfering component can dissolve in one mL of extraction solution, for example, greater than about 50 mg of the interfering component can dissolve in one mL of extraction solution, greater than about 100 mg of the interfering component can dissolve in one mL of extraction solution, greater than about 200 mg of the interfering component can dissolve in one mL of extraction solution, greater than about 350 mg of the interfering component can dissolve in one mL of extraction solution, greater than about 500 mg of the interfering component can dissolve in one mL of extraction solution, greater than about 1000 mg of the interfering component can dissolve in one mL of extraction solution, greater than about 1500 mg of the interfering component can dissolve in one mL of extraction solution, greater than about 1800 mg of the interfering component can dissolve in one mL of extraction solution; or greater than about 2000 mg of the interfering component can dissolve in one mL of extraction solution.

Bazedoxifene acetate or one or more other components is identified as substantially contained in, e.g., a filtrand, filtrate, or supernatant solution or solid produced by centrifugation. The term “substantially contained” as used herein means that at least about 70% by weight of the identified bazedoxifene acetate or other component is contained in the specified filtrate, filtrand, supernatant solution or solid compared to the amount of the corresponding component contained in the initial pharmaceutical composition being analyzed. In some instances, at least about 80% or at least 90% of the identified bazedoxifene acetate or other component is contained in the specified filtrate, filtrand, supernatant solution or solid.

The term “substantially free” as used herein means that the interfering component makes up no more than about 25% by weight of the final composition as prepared according to a method described herein. In certain embodiments, the interfering component makes up no more than about 20%, about 10%, about 5%, or about 1% by weight of the final composition.

In certain embodiments, the extraction medium is a solution comprising one or more acetate salts. In certain embodiments, the solution includes ammonium acetate, sodium acetate, potassium acetate, magnesium acetate, calcium acetate, or mixtures thereof. In certain embodiments, the solution includes ammonium acetate, sodium acetate, or mixtures thereof. In some cases, the solution includes sodium acetate. Certain embodiments provide methods to extract bazedoxifene acetate from formulations, without altering the bazedoxifene acetate crystal form. While not to be bound by theory, it is believed that high concentrations of acetate (Ac⁻) may suppress the dissociation and dissolution of bazedoxifene acetate.

In certain embodiments, the solution has a concentration of about 0.05 M to about 1 M with respect to acetate. In certain embodiments, the solution has a concentration of about 0.1 M to about 0.9 M with respect to acetate. In some embodiments, the solution has a concentration of about 0.15 M to about 0.85 M with respect to acetate. In some embodiments, the solution has a concentration of about 0.2 M to about 0.8 M with respect to acetate. In some embodiments, the solution has a concentration of about 0.25 M to about 0.75 M with respect to acetate. In some embodiments, the solution has a concentration of about 0.3 M to about 0.7 M with respect to acetate. In some embodiments, the solution has a concentration of about 0.35 M to about 0.65 M with respect to acetate. In some embodiments, the solution has a concentration of about 0.4 M to about 0.6 M with respect to acetate. In some embodiments, the solution has a concentration of about 0.45 M to about 0.55 M with respect to acetate. In some embodiments, the solution has a concentration of about 0.5 M with respect to acetate.

In some embodiments, the extraction medium has a pH of about 1 to about 13. In some embodiments, the extraction medium has a pH of about 5 to about 12. In certain embodiments, the solution has a pH of about 5 to about 10. In some embodiments, the extraction medium has a pH of about 6 to about 11. In some embodiments, the extraction medium has a pH of about 6.5 to about 10.5. In certain embodiments, the extraction medium has a pH of about 6 to about 9.2. In some embodiments, the extraction medium has a pH of about 6.2 to about 8.5. In some embodiments, the extraction medium has a pH of about 7 to about 10. In some embodiments, the extraction medium has a pH of about 7.5 to about 9.5. In some embodiments, the extraction medium has a pH of about 8 to about 9. In some embodiments, the extraction medium has a pH of about 8.2 to about 8.5. In some embodiments, the extraction medium has a pH of about 8.3 to about 8.4.

In some embodiments, the extraction medium includes a solution of ammonium acetate. In some embodiments, the extraction medium includes about 0.5 M ammonium acetate. In some embodiments, the extraction medium includes about 0.5 M ammonium acetate and has a pH of about 6.2.

In some embodiments, the extraction medium includes a mixture of ammonium acetate and sodium acetate. In some embodiments, the extraction medium includes a solution of: about 0.05 M to about 0.5 M ammonium acetate; and about 0.05 M to about 0.5 M sodium acetate. In some embodiments, the extraction medium includes ammonium acetate and sodium acetate and has a pH of about 6.5 to about 7.5.

In some embodiments, the extraction medium includes about 0.1 M ammonium acetate and about 0.4 M sodium acetate. In some embodiments, the extraction medium includes about 0.15 M ammonium acetate and about 0.35 M sodium acetate. In some embodiments, the extraction medium includes about 0.2 M ammonium acetate and about 0.3 M sodium acetate. In some embodiments, the extraction medium includes about 0.25 M ammonium acetate and about 0.25 M sodium acetate. In some embodiments, the extraction medium includes about 0.3 M ammonium acetate and about 0.2 M sodium acetate. In some embodiments, the extraction medium includes about 0.35 M ammonium acetate and about 0.15 M sodium acetate. In some embodiments, the extraction medium includes about 0.4 M ammonium acetate and about 0.1 M sodium acetate. In some embodiments, the extraction medium includes about 0.45 M ammonium acetate and about 0.05 M sodium acetate.

In some embodiments, the extraction medium includes about 0.125 M ammonium acetate and about 0.375 M sodium acetate. In some embodiments, the extraction medium includes about 0.125 M ammonium acetate and about 0.375 M sodium acetate has a pH of about 6.85. In some embodiments, the extraction medium includes about 0.05 M ammonium acetate and about 0.45 M sodium acetate. In some embodiments, the extraction medium includes about 0.05 M ammonium acetate and about 0.45 M sodium acetate has a pH of about 7.18.

In some embodiments, the extraction medium includes sodium acetate. In some embodiments, the extraction medium includes about 0.5 M sodium acetate. In some embodiments, the extraction medium includes about 0.5 M sodium acetate and has a pH of about 8.34.

In certain embodiments, the pharmaceutical composition is provided as at least one unit dosage form. Examples of unit dosage forms include, without limitation, tablet, capsule, gel cap, buccal form, troche, and lozenge. In certain embodiments, the unit dosage form is a tablet. In certain embodiments, the dosage units described herein can utilize standard delay or time release formulations or spansules.

In certain embodiments, any coating is removed from the dosage unit prior to contacting it with the extraction medium. In some embodiments, the pharmaceutical dosage unit is broken apart prior to and/or during mixing with the extraction medium.

In certain embodiments, the amount of the extraction medium used during the contacting of the pharmaceutical composition with the extraction medium to produce the suspension is from about 0.2 ml per dosage unit to about 10 ml per dosage unit (e.g., tablet). In some embodiments, the amount of the extraction medium used during the mixing of the pharmaceutical composition and the extraction medium is about 0.8 ml per dosage unit. In some embodiments, the amount of the extraction medium used during the mixing of the pharmaceutical composition and the extraction medium is about 1 ml per dosage unit. In some embodiments, the amount of the extraction medium used during the mixing of the pharmaceutical composition and the extraction medium is about 1.67 ml per dosage unit. In some embodiments, the amount of the extraction medium used during the mixing of the pharmaceutical composition and the extraction medium is about 2.5 ml per dosage unit. In some embodiments, the amount of extraction medium used is inversely related to the molar concentration of acetate ion in the extraction medium.

In certain embodiments, the pharmaceutical composition is contacted with the extraction medium for about 1 to about 120 minutes. In some embodiments, the pharmaceutical composition is contacted with the extraction medium for about 5 to about 30 minutes. In some embodiments, the pharmaceutical composition is contacted with the extraction medium for about 5 to about 10 minutes. In some embodiments, the pharmaceutical composition is contacted with the extraction medium for about 2 to about 5 minutes. In some embodiments, the pharmaceutical composition is contacted with the extraction medium for about 2 minutes.

In some embodiments, a mixture of the pharmaceutical composition and the extraction medium is filtered to produce a filtrand. In some instances, washing is performed with additional extraction medium. In some embodiments, the filtrand is dried. In some embodiments, the filtrand is dried overnight. In some embodiments, the filtrand is dried at an elevated temperature, for example, at about 40° C. In some embodiments, the filtrand is dried at an elevated temperature, for example, at about 40° C. overnight. In some embodiments, the filtrand is dried at room temperature. In some embodiments, the filtrand is dried under vacuum. In some embodiments, the filtrand is dried under vacuum and at an elevated temperature, for example, at about 40° C.

Another aspect provides methods of separating a pharmaceutical composition comprising bazedoxifene acetate Form A and/or Form B and one or more components (e.g., lactose) that produce X-Ray diffraction patterns having one or more interfering peaks at or near the characteristic peak or peaks for bazedoxifene acetate Form A and/or Form B. The method includes: (a) contacting the pharmaceutical composition with a solution comprising at least one acetate salt to produce a suspension, in which the bazedoxifene acetate Form A and/or Form B is substantially insoluble in the solution and in which the one or more components (e.g., lactose) are substantially soluble in the solution; (b) filtering the suspension to produce a filtrate and a filtrand, in which the one or more components are substantially contained in the filtrate; and (c) washing and drying the filtrand to produce a composition substantially free of the one or more components (e.g., lactose) that produce X-Ray diffraction patterns having one or more interfering peaks.

In certain embodiments, the pharmaceutical composition is provided as tablet form, and the method further includes removing any coating from the tablet prior to contacting the tablet with the solution.

In certain embodiments, the one or more components that produce X-Ray diffraction patterns having interfering peaks include, without limitation, lactose, sucrose, or mixtures thereof

In certain embodiments, the characteristic peak for bazedoxifene acetate Form A is at about 12.8±0.2° in 2theta angular degree by Cu radiation. In certain embodiments, the characteristic peaks for bazedoxifene acetate Form B are at about 12.0±0.2° and 13.3±0.2° in 2theta angular degree by Cu radiation. In some embodiments, the interfering peaks are at from about 11.6±0.2° to about 13.7±0.2° in 2theta angular degree by Cu radiation.

Yet another aspect provides a method of detecting bazedoxifene acetate Form A and/or Form B in a pharmaceutical composition comprising bazedoxifene acetate Form A and/or Form B and one or more components that produce X-Ray diffraction patterns having one or more interfering peaks at or near the characteristic peak or peaks for bazedoxifene acetate Form A and/or Form B. The method includes: producing a composition containing bazedoxifene acetate Form A and/or Form B by a method as described hereinabove, wherein the composition is substantially free of the one or more components that produce X-Ray diffraction patterns having one or more interfering peaks; forming the composition containing bazedoxifene acetate Form A and/or Form B into powder or tablet or pellet for X-Ray diffraction measurement; and analyzing the powder or tablet or pellet using X-Ray diffraction.

A further aspect provides a method of separating a pharmaceutical composition comprising bazedoxifene acetate and one or more components that produce X-Ray diffraction patterns having one or more interfering peaks at or near the characteristic peak or peaks for bazedoxifene acetate. The method includes: (a) contacting the pharmaceutical composition with an extraction medium to produce a suspension, in which the bazedoxifene acetate is substantially insoluble in the extraction medium and in which the one or more components are substantially soluble in the extraction medium; (b) centrifuging the suspension to produce a solid and a supernatant solution, in which the one or more components are substantially contained in the supernatant solution; and (c) collecting and drying the solid to produce a composition substantially free of the one or more components that produce X-Ray diffraction patterns having one or more interfering peaks.

In certain embodiments, the pharmaceutical composition further includes conjugated estrogens. In certain embodiments, the pharmaceutical composition includes a core containing conjugated estrogens and an outer layer containing bazedoxifene acetate. In certain embodiments, the contacting of the pharmaceutical composition with an extraction medium in step (a) is stopped when a filler coating between the conjugated estrogen core and the bazedoxefene outer layer is exposed.

In certain embodiments, the bazedoxifene acetate is substantially contained in the solid produced by centrifugation. In some embodiments, step (b) is repeated as needed. In some embodiments, the method further includes removing the supernatant solution provided in step (b). In some instances, the supernatant solution can be removed by decanting or through pipette. In some embodiments, the method further includes washing the solid provided in step (b).

In certain embodiments, the solid is collected by filtration. In certain embodiments, the method further includes forming the composition obtained in step (c) into powder or a tablet or pellet for X-Ray diffraction measurement. In some embodiments, the method further includes analyzing the composition obtained in step (c) or the powder, tablet or pellet prepared from the composition using X-Ray diffraction.

In certain embodiments, the bazedoxifene acetate is bazedoxifene acetate Form A and/or bazedoxifene acetate Form B.

In certain embodiments, the characteristic peak for bazedoxifene acetate Form A is at about 12.8±0.2° in 2theta angular degree by Cu radiation, and the characteristic peaks for bazedoxifene acetate Form B are at about 12.0±0.2° and about 13.3±0.2° in 2theta angular degree by Cu radiation.

In certain embodiments, the one or more components that produce X-Ray diffraction patterns having interfering peaks include, without limitation, pharmaceutically acceptable diluents, fillers, excipients, binding agents, lubricants, disintegrants, suspending or stabilizing agents, and mixtures thereof. In certain embodiments, the one or more components that produce X-Ray diffraction patterns having interfering peaks include lactose, sucrose, or mixtures thereof. In some embodiments, the one or more components that produce X-Ray diffraction patterns having interfering peaks include sucrose.

In certain embodiments, the interfering peaks are at from about 11.9±0.2° to about 13.3±0.2° in 2theta angular degree by Cu radiation.

In certain embodiments, the extraction medium is a solution comprising one or more acetate salts. In certain embodiments, the solution includes ammonium acetate, sodium acetate, potassium acetate, magnesium acetate, calcium acetate, or mixtures thereof. In certain embodiments, the solution includes ammonium acetate, sodium acetate, or mixtures thereof.

In certain embodiments, the solution has a concentration of about 0.05 M to about 1 M with respect to acetate. In certain embodiments, the solution has a concentration of about 0.1 M to about 0.9 M with respect to acetate. In some embodiments, the solution has a concentration of about 0.15 M to about 0.85 M with respect to acetate. In some embodiments, the solution has a concentration of about 0.2 M to about 0.8 M with respect to acetate. In some embodiments, the solution has a concentration of about 0.25 M to about 0.75 M with respect to acetate. In some embodiments, the solution has a concentration of about 0.3 M to about 0.7 M with respect to acetate. In some embodiments, the solution has a concentration of about 0.35 M to about 0.65 M with respect to acetate. In some embodiments, the solution has a concentration of about 0.4 M to about 0.6 M with respect to acetate. In some embodiments, the solution has a concentration of about 0.45 M to about 0.55 M with respect to acetate. In some embodiments, the solution has a concentration of about 0.5 M with respect to acetate.

In some embodiments, the extraction medium has a pH of about 1 to about 13. In some embodiments, the extraction medium has a pH of about 5 to about 12. In certain embodiments, the extraction medium has a pH of about 5 to about 10. In some embodiments, the extraction medium has a pH of about 6 to about 11. In some embodiments, the extraction medium has a pH of about 6.5 to about 10.5. In certain embodiments, the extraction medium has a pH of about 6 to about 9.2. In some embodiments, the extraction medium has a pH of about 6.2 to about 8.5. In some embodiments, the extraction medium has a pH of about 7 to about 10. In some embodiments, the extraction medium has a pH of about 7.5 to about 9.5. In some embodiments, the extraction medium has a pH of about 8 to about 9. In some embodiments, the extraction medium has a pH of about 8.2 to about 8.5. In some embodiments, the extraction medium has a pH of about 8.3 to about 8.4.

In certain embodiments, the pharmaceutical composition is provided as at least one unit dosage form. Non-limiting examples of the unit dosage form include tablet, capsule, gel cap, buccal form, troche, and lozenge. In certain embodiments, the pharmaceutical composition is provided as a tablet. In some embodiments, the tablet includes a core and an outer layer. In some embodiments, the core contains conjugated estrogens and the outer layer contains bazedoxifene acetate.

In certain embodiments, the amount of the extraction medium used during the contacting of the pharmaceutical composition with the extraction medium to produce the suspension is from about 0.2 ml per dosage unit (e.g., tablet) to about 10 ml per dosage unit. In some embodiments, the amount of the extraction medium used during the mixing of the pharmaceutical composition and the extraction medium is about 0.8 ml per dosage unit. In some embodiments, the amount of the extraction medium used during the mixing of the pharmaceutical composition and the extraction medium is about 1 ml per dosage unit. In some embodiments, the amount of the extraction medium used during the mixing of the pharmaceutical composition and the extraction medium is about 1.67 ml per dosage unit. In some embodiments, the amount of the extraction medium used during the mixing of the pharmaceutical composition and the extraction medium is about 2.5 ml per dosage unit. In some embodiments, the amount of extraction medium used is inversely related to the molar concentration of acetate ion in the extraction medium.

In certain embodiments, the pharmaceutical composition is contacted with the extraction medium for about 1 to about 120 minutes. In some embodiments, the pharmaceutical composition is contacted with the extraction medium for about 5 to about 30 minutes. In some embodiments, the pharmaceutical composition is contacted with the extraction medium for about 5 to about 10 minutes. In some embodiments, the pharmaceutical composition is contacted with the extraction medium for about 2 to about 5 minutes in some embodiments, the pharmaceutical composition is contacted with the extraction medium for about 2 minutes.

Another aspect provides a method of separating a pharmaceutical composition comprising bazedoxifene acetate Form A and/or Form B and one or more components that produce X-Ray diffraction patterns having one or more interfering peaks at or near the characteristic peak or peaks for bazedoxifene acetate Form A and/or Form B. The method includes: (a) contacting the pharmaceutical composition with a solution comprising at least one acetate salt to produce a suspension, wherein bazedoxifene acetate Form A and/or Form B is substantially insoluble in the solution and wherein the one or more components are substantially soluble in the solution; (b) centrifuging the suspension to produce a solid and a supernatant solution, wherein the one or more components are substantially contained in the supernatant solution; and (c) collecting and drying the solid to produce a composition substantially free of the one or more components that produce X-Ray diffraction patterns having one or more interfering peaks.

In certain embodiments, the pharmaceutical composition is provided as a tablet. In some embodiments, the tablet includes a core and an outer layer. In some instances, the core contains conjugated estrogens and the outer layer contains bazedoxifene acetate Form A and/or Form B. In certain embodiments, the method further includes removing any coating from the tablet prior to contacting the tablet with the solution.

In certain embodiments, the one or more components that produce X-Ray diffraction patterns having interfering peaks include lactose, sucrose, or mixtures thereof. In certain embodiments, the one or more components that produce X-Ray diffraction patterns having interfering peaks include sucrose.

In certain embodiments, the characteristic peak for bazedoxifene acetate Form A is at about 12.8±0.2° in 2theta angular degree by Cu radiation. In certain embodiments, the characteristic peaks for bazedoxifene acetate Form B are at about 12.0±0.2° and 13.3±0.2° in 2theta angular degree by Cu radiation. In some embodiments, the interfering peaks are at from about 11.9±0.2° to about 13.3±0.2° in 2theta angular degree by Cu radiation.

A further aspect provides a method of detecting bazedoxifene acetate Form A and/or Form B in a pharmaceutical composition comprising bazedoxifene acetate Form A and/or Form B and one or more components (e.g., sucrose) that produce X-Ray diffraction patterns having one or more interfering peaks at or near the characteristic peak or peaks for bazedoxifene acetate Form A and/or Form B. The method includes:

(a) producing a composition containing bazedoxifene acetate Form A and/or Form B by a method as described herein above, wherein the composition is substantially free of the one or more components (e.g., sucrose) that produce X-Ray diffraction patterns having one or more interfering peaks;

(b) forming the composition containing bazedoxifene acetate Form A and/or Form B into a tablet or pellet for X-Ray diffraction measurement; and

(c) analyzing the tablet or pellet using X-Ray diffraction.

In some embodiments, the pharmaceutical composition comprising bazedoxifene acetate and one or more components that produce XRD patterns having interfering peaks at or near the characteristic peaks of bazedoxifene acetate includes a mixture of bazedoxifene acetate Form A and bazedoxifene acetate Form B.

In some embodiments, the methods described herein includes: preparing a final composition that is substantially free of interfering components (e.g., lactose or sucrose) for XRD analysis. In some embodiments, the final composition is ground and pressed into a tablet or pellet for XRD measurement. In some embodiments, the analysis of the final composition is carried out using a Phillips X-Pert PW3040-MPD diffractometer. In some embodiments, the analysis of the final composition is carried out using a Bruker D8 Discover X-ray diffractometer with GADDS.

The embodiments of the methods described above can be combined in any manner. Thus, features from one embodiment can be combined with features from any other embodiment. For example, the embodiments described above can be combined in a manner to produce methods of detecting bazedoxifene acetate Form A and/or Form B in a pharmaceutical composition comprising bazedoxifene acetate and one or more components that produce XRD patterns having interfering peaks at or near the characteristic peaks of bazedoxifene acetate Form A and/or Form B, comprising: removing any coating from at least one dosage unit comprising bazedoxifene acetate and one or more pharmaceutically acceptable diluents, fillers, excipients, binding agents, lubricants, disintegrants, or suspending or stabilizing agents (e.g., lactose); adding sodium acetate solution (about 0.5 M) to the pharmaceutical composition to produce a suspension with stirring for about 2 to about 15 minutes; filtering and washing the suspension with additional sodium acetate solution to produce a filtrand; drying the filtrand; grinding and pressing the filtrand into pellets for XRD measurement; and analyzing the prepared pellets using an XRD diffractometer, e.g., Bruker D8 Discover X-ray diffractometer with GADDS.

Furthermore, the embodiments described above can be combined in a manner to produce methods of detecting bazedoxifene acetate Form A and/or Form B in a pharmaceutical composition comprising bazedoxifene acetate and one or more components that produce XRD patterns having interfering peaks at or near the characteristic peaks of bazedoxifene acetate Form A and/or Form B. The method includes: removing any coating from at least one dosage unit comprising bazedoxifene acetate and one or more pharmaceutically acceptable diluents, fillers, excipients, binding agents, lubricants, disintegrants, or suspending or stabilizing agents (e.g., sucrose); adding an appropriate amount of sodium acetate solution (about 0.2 M) to the pharmaceutical composition to produce a suspension with stirring for about 2 to about 10 minutes; centrifuging the suspension to produce a solid and a supernatant solution; removing the supernatant solution; adding additional sodium acetate solution to the solid to produce another suspension with shaking; centrifuging the suspension to produce another solid and another supernatant solution; removing the supernatant solution; filtering and drying the solid; grinding and pressing the solid into pellets for XRD measurement; and analyzing the prepared pellets using an XRD diffractometer, e.g., Bruker D8 Discover X-ray diffractometer with GADDS.

The methods disclosed according to certain embodiments herein advantageously allow for detecting bazedoxifene acetate Form B that is present in a pharmaceutical composition in low levels. For example, about 2% by weight of bazedoxifene acetate Form B relative to the total bazedoxifene acetate can be detected after the application of the extraction procedure as described according to certain embodiments herein due to the increase of the detectable bazedoxifene acetate signals in X-Ray diffraction upon removal of interfering component(s).

The following examples are for illustrative purposes only and are not to be construed as limiting. The tablets used in the examples can be prepared by a method described in, e.g., WO 02/03987, U.S. Pat. No. 6,479,535, US 2007/0003623, and patent application Ser. No. 11/946,586, filed on Nov. 28, 2007, and U.S. Ser. No. 12/013,109, filed on Jan. 11, 2008.

### EXAMPLES

### Example 1

### Extraction Procedure for Tablets Containing Bazedoxifene Acetate (BZA)

Tablets used herein comprise BZA Form A, lactose monohydrate, ascorbic acid, microcrystalline cellulose, pregelatinized starch, sodium starch glycolate, colloidal silicon dioxide, magnesium stearate, and sodium lauryl sulfate. The tablets further comprise a coating containing Opadry® White and Opadry® Clear.

The coatings of three of the tablets were removed. The resulting tablets were then placed in a small beaker. About 3.0 ml of extraction medium solution was added to the beaker to produce a mixture. The mixture was broken up and stirred for about 5 to about 15 minutes. The resulting mixture was filtered and washed with an additional 3.0 ml of extraction medium solution three times to produce a solid (filtrand). The recovered solid was dried at about 40° C. overnight. The dried solid was grinded and pressed into pellets for XRD measurement. This procedure can be scaled up or down for various quantities of BZA tablets. This procedure was employed for the samples found in Table 1.

### Example 2

### Extraction Procedure for Tablets Containing Bazedoxifene Acetate (BZA) and Conjugated Estrogens (CE)

Tablets used herein comprise a core comprising CE, lactose monohydrate, microcrystalline cellulose, and hypromellose; an outer layer comprising BZA Form A, ascorbic acid, sucrose, hypromellose, and sucrose palmitic acid ester; and a filler coat between the core and the outer layer, which filler coat containing sucrose, microcrystalline cellulose, hypromellose, polyethylene glycol. The tablets further comprise a coating containing Opadry® Pink and Opadry® 2 Clear.

The coatings of twenty of the BZA/CE tablets were removed by razor blades or other appropriate means. The resulting tablets were placed in a 50 ml beaker. About 20-25 ml of 0.2 M sodium acetate solution was added to the beaker to produce a mixture. The mixture was stirred with a magnetic stirring bar to produce a suspension. The resulting suspension was transferred into a 50 ml centrifuge tube. About another 10-25 ml of 0.2 M sodium acetate solution was added to the beaker containing residual tablets to produce a mixture. The mixture was stirred to produce a suspension, which was again transferred into the same centrifuge tube. (This step may be repeated as needed until a filler coating between the CE core and the BZA outer layer was exposed. The filler coating between the BZA outer layer and the CE core usually looks smooth and whitish.) The resulting suspension was centrifuged to produce a supernatant solution and a solid. The supernatant solution was removed. The solid remained in the centrifuge tube was added about 40 ml of 0.2 M sodium acetate, then shaken, centrifuged and the resulting supernatant solution removed again. (This step may be repeated as needed.) The remaining solid in the centrifuge tube was filtered to remove any leftover extraction medium. The recovered solid produced by filtration was dried at about 40° C. overnight (12 to 18 hours).

The dried solid was grinded and pressed into pellets using an IR hydraulic presser for XRD measurement.

### Example 3

### X-Ray Powder Diffraction

X-Ray Powder Diffraction analyses were carried out on samples prepared according to Examples 1 and 2 using a Bruker D8 Discover X-ray diffractometer with GADDS. The diffractometer power was set at 40 kV and 40 mA. The collimator diameter of the instrument was about 0.8 mm and the detector-to-sample distance was set at about 30 cm. The X-ray incident angle relative to the pellet/tablet surface was about 40 and the detection angle relative to the pellet/tablet surface was about 16±0.2°. Data were collected at about 120 minutes to about 240 minutes.

Alternatively, X-Ray Diffraction analyses on powder samples can be carried out on a (Philips X'Pert MPD) X-ray diffractometer using Cu radiation x-ray beams. The diffractometer power was set at 40 kV and 40 mA. A continuous scan at 0.02 degree/second from 4 to 40° was used. FIGS. 1 and 2 show the XRD patterns collected from bazedoxifene acetate Form A and Form B, lactose and sucrose powders.

### Example 4

### Bazedoxifene Acetate (BZA) Solubility Study

To examine potential bazedoxifene acetate loss during extraction, a bazedoxifene acetate solubility study was performed. Tablets (same as those used in Example 1) were placed in an extraction medium (0.5 M of sodium acetate). The solution part was tested by High Pressure Liquid Chromatography (HPLC) to determine how much bazedoxifene acetate had dissolved in the extraction medium. Known amount of BZA was dissolved in and diluted with acetonitrile/water (1:1) into different concentrations (standard bazedoxifene acetate solutions), and then chromatographed on a reversed phase C18 column to provide a standard chromatogram as a reference. A calibration curve between the bazedoxifene concentration and the UV peak area at 220 nm was established from these standard bazedoxifene acetate solutions. Bazedoxifene acetate concentration of a given sample is determined by its UV peak area at 220nm using the calibration curve . The identification of bazedoxifene by HPLC is determined by comparing the retention time of the bazedoxifene peak in the sample preparation chromatogram to that of the bazedoxifene peak in the standard preparation. HPLC Column: C18. 5 μm, 150×4.6 mm; detector wavelength: 220 nm; flow rate: about 1.5 mL per minute; injection volume: 10 μL; mobile phase: constant 68:32 (v/v)−25 mM phosphate buffer, pH 3.0: acetonitrile; run time: about 10 minutes.

Less than 0.5% of bazedoxifene acetate by weight dissolved in 2 hours or longer. Since the extraction procedures typically contact the bazedoxifene acetate formulation with the extraction medium for less than about two hours, for example, less than about half an hour, bazedoxifene acetate loss and solution-mediated transformation during the extraction procedure are typically not significant.

### Example 5

### Bazedoxifene acetate Recovery in Different Extraction Media

The following table illustrates the results of experiments using the extraction methods of Example 1. The extractions were performed using different extraction media and the results were measured in terms of peak area of bazedoxifene using XRD as described in Example 3. As can be seen from Table 1, the extraction medium with the highest recovery rate for this particular set of experiments was 0.50 M Sodium Acetate, at a pH of about 8.34.

TABLE 1
Bazedoxifene acetate Recovery in Different Extraction Media

| | | Amount of | Peak Area of |
|---|---|---|---|
| | Extraction Solution, | solution used | Bazedoxifene |
| Experiment | pH | (mL/tablet) | (count · degree)* |
| 1 | Millipore Water, pH 5.9 | 2.50 | 3.3 |
| 2 | Millipore Water, pH 5.9 | 1.67 | 4.9 |
| 3 | Diluted Acetic Acid, pH | 2.50 | No Detection |
| | 1.96 | | |
| 4 | Diluted Acetic Acid, pH | 0.83 | Lactose |
| | 1.96 | | Detected |
| 5 | Millipore Water, pH 5.9 | 0.83 | 11.6 |
| 6 | 0.50 M Ammonium | 1.0 | 22.7 |
| | Acetate, pH 6.20 | | |
| 7 | 0.125 M Ammonium | 1.0 | 23.1 |
| | Acetate + 0.375 M | | |
| | Sodium Acetate, pH | | |
| | 6.85 | | |
| 8 | 0.05 M Ammonium | 1.0 | 32.0 |
| | Acetate + | | |
| | 0.45 M Sodium | | |
| | Acetate, pH 7.18 | | |
| 9 | 0.50 M Sodium Acetate, | 1.0 | 36.2 |
| | pH 8.34 | | |
| 10 | 0.50 M Sodium Chloride | 1.0 | 21.8 |
| | pH 9.20 adjusted by | | |
| | Sodium Hydroxide | | |

*Peak at 12.8° for bazedoxifene acetate Form A is used to indicate the bazedoxifene acetate recovery.

Various modifications, in addition to those described herein, will be apparent to those skilled in the art from the foregoing description. Such modifications are also intended to fall within the scope of the appended claims. Each patent, patent application and literature referenced in the present application is incorporated herein by reference in its entirety.

What is claimed is:

1. A method of separating a pharmaceutical composition comprising bazedoxifene acetate and one or more components that produce X-Ray diffraction patterns having one or more interfering peaks at or near the characteristic peak or peaks for bazedoxifene acetate, said method comprising:
(a) contacting said pharmaceutical composition with an extraction medium to produce a suspension, wherein bazedoxifene acetate is substantially insoluble in the extraction medium and wherein said one or more components are substantially soluble in the extraction medium;
(b) filtering said suspension to produce a filtrate and a filtrand, wherein said one or more components are substantially contained in said filtrate; and
(c) drying the filtrand to obtain a composition substantially free of said one or more components that produce X-Ray diffraction patterns having one or more interfering peaks.

2. The method of claim 1, wherein said bazedoxifene acetate is substantially contained in said filtrand.

3. The method of claim 1, further comprising washing the filtrand.

4. The method of claim 1, further comprising forming the composition obtained in step (c) into a tablet or pellet for X-Ray diffraction measurement.

5. The method of claim 1, further comprising analyzing the composition obtained in step (c) or a tablet or pellet prepared from the composition using X-Ray diffraction.

6. The method of claim 1, wherein said bazedoxifene acetate is bazedoxifene acetate Form A and/or bazedoxifene acetate Form B.

7. The method of claim 6, wherein the characteristic peak for bazedoxifene acetate Form A is at about 12.8±0.2° in 2theta angular degree by Cu radiation, and the characteristic peaks for bazedoxifene acetate Form B are at about 12.0±0.2° and 13.3±0.2° in 2theta angular degree by Cu radiation.

8. The method of claim 1, wherein said one or more components that produce X-Ray diffraction patterns having interfering peaks include pharmaceutically acceptable diluents, fillers, excipients, binding agents, lubricants, disintegrants, suspending or stabilizing agents, or mixtures thereof.

9. The method of claim 8, wherein said one or more components that produce X-Ray diffraction patterns having interfering peaks include lactose, sucrose, or mixtures thereof.

10. The method of claim 1, wherein said interfering peaks are at from about 11.6±0.2° to about 13.7±0.2° in 2theta angular degree by Cu radiation.

11. The method of claim 1, wherein said extraction medium is a solution comprising one or more acetate salts.

12. The method of claim 11, wherein said solution comprises ammonium acetate, sodium acetate, potassium acetate, magnesium acetate, calcium acetate, or a mixture thereof.

13. The method of claim 11, wherein said solution comprises ammonium acetate, sodium acetate, or a mixture thereof.

14. The method of claim 11, wherein said solution has a concentration of about 0.05 M to about 1 M with respect to acetate.

15. The method of claim 11, wherein said solution has a concentration of about 0.25 M to about 0.75 M with respect to acetate.

16. The method of claim 11, wherein said solution has a concentration of about 0.45 M to about 0.55 M with respect to acetate.

17. The method of claim 11, wherein said solution has a pH between about 5 and about 10.

18. The method of claim 11, wherein said solution has a pH between about 6 and about 9.2.

19. The method of claim 11, wherein said solution has a pH between about 6.2 and about 8.5.

20. The method of claim 1, wherein said pharmaceutical composition is provided as at least one unit dosage form, wherein the unit dosage form is a tablet, capsule, gel cap, buccal form, troche, or lozenge.

21. The method of claim 20, further comprising removing any coating from said unit dosage form prior to contacting the unit dosage form with said extraction medium.

22. The method of claim 1, wherein the amount of said extraction medium used during the contacting of said pharmaceutical composition with said extraction medium to produce the suspension is from about 0.2 ml per unit dosage form to about 10 ml per unit dosage form.

23. The method of claim 1, wherein said pharmaceutical composition is contacted with said extraction medium for about 1 minute to about 120 minutes.

24. The method of claim 1, wherein said pharmaceutical composition is contacted with said extraction medium for about 5 minutes to about 30 minutes.

25. The method of claim 1, wherein said pharmaceutical composition is contacted with said extraction medium for about 5 minutes to about 15 minutes.

26. A method of separating a pharmaceutical composition comprising bazedoxifene acetate Form A and/or Form B and one or more components that produce X-Ray diffraction patterns having one or more interfering peaks at or near the characteristic peak or peaks for bazedoxifene acetate Form A and/or Form B, said method comprising:
(a) contacting said pharmaceutical composition with a solution comprising at least one acetate salt to produce a suspension, wherein bazedoxifene acetate Form A and/or Form B is substantially insoluble in the solution and wherein said one or more components are substantially soluble in the solution;
(b) filtering said suspension to produce a filtrate and a filtrand, wherein said one or more components are substantially contained in said filtrate; and
(c) washing and drying the filtrand to produce a composition substantially free of said one or more components that produce X-Ray diffraction patterns having one or more interfering peaks.

27. The method of claim 26, wherein said pharmaceutical composition is provided as a tablet, and further comprising removing any coating from said tablet prior to contacting the tablet with said solution.

28. The method of claim 26, wherein said one or more components that produce X-Ray diffraction patterns having interfering peaks include lactose, sucrose, or a mixture thereof.

29. The method of claim 26, wherein the characteristic peak for bazedoxifene acetate Form A is at about 12.8±0.2° in 2theta angular degree by Cu radiation.

30. The method of claim 26, wherein the characteristic peaks for bazedoxifene acetate Form B are at about 12.0±0.2° and 13.3±0.2° in 2theta angular degree by Cu radiation.

31. The method of claim 26, wherein said interfering peaks are at from about 11.6±0.2° to about 13.7±0.2° in 2theta angular degree by Cu radiation.

32. A method of detecting bazedoxifene acetate Form A and/or Form B in a pharmaceutical composition comprising bazedoxifene acetate Form A and/or Form B and one or more components that produce X-Ray diffraction patterns having one or more interfering peaks at or near the characteristic peak or peaks for bazedoxifene acetate Form A and/or Form B, said method comprising:
(a) producing a composition containing bazedoxifene acetate Form A and/or Form B by the method of claim 1, wherein the composition is substantially free of said one or more components that produce X-Ray diffraction patterns having one or more interfering peaks;
(b) forming the composition containing bazedoxifene acetate Form A and/or Form B into a tablet or pellet for X-Ray diffraction measurement; and
(c) analyzing said tablet or pellet using X-Ray diffraction.

33. A method of separating a pharmaceutical composition comprising bazedoxifene acetate and one or more components that produce X-Ray diffraction patterns having one or more interfering peaks at or near the characteristic peak or peaks for bazedoxifene acetate, said method comprising:
(a) contacting said pharmaceutical composition with an extraction medium to produce a suspension, wherein bazedoxifene acetate is substantially insoluble in the extraction medium and wherein said one or more components are substantially soluble in the extraction medium;
(b) centrifuging said suspension to produce a solid and a supernatant solution, wherein said one or more components are substantially contained in said supernatant solution; and
(c) collecting and drying said solid to produce a composition substantially free of said one or more components that produce X-Ray diffraction patterns having one or more interfering peaks.

34. The method of claim 33, wherein the pharmaceutical composition further comprises conjugated estrogens.

35. The method of claim 33, wherein said bazedoxifene acetate is substantially contained in said solid produced in step (b).

36. The method of claim 33, further comprising removing said supernatant solution produced in step (b).

37. The method of claim 33, further comprising washing the solid in step (b).

38. The method of claim 33, wherein the collecting of said solid is achieved through filtration.

39. The method of claim 33, further comprising forming the composition obtained in step (c) into a tablet or pellet for X-Ray diffraction measurement.

40. The method of claim 33, further comprising analyzing the composition obtained in step (c) or the tablet or pellet prepared from the composition using X-Ray diffraction.

41. The method of claim 33, wherein said bazedoxifene acetate is bazedoxifene acetate Form A and/or bazedoxifene acetate Form B.

42. The method of claim 33, wherein the characteristic peak for bazedoxifene acetate Form A is at about 12.8±0.2° in 2theta angular degree by Cu radiation, and the characteristic peaks for bazedoxifene acetate Form B are at about 12.0±0.2° and about 13.3±0.2° in 2theta angular degree by Cu radiation.

43. The method of claim 33, wherein said one or more components that produce X-Ray diffraction patterns having interfering peaks include pharmaceutically acceptable diluents, fillers, excipients, binding agents, lubricants, disintegrants, suspending or stabilizing agents, or mixtures thereof.

44. The method of claim 33, wherein said one or more components that produce X-Ray diffraction patterns having interfering peaks include lactose, sucrose, or a mixture thereof.

45. The method of claim 33, wherein said interfering peaks are at from about 11.9±0.2° to about 13.3±0.2° in 2theta angular degree by Cu radiation.

46. The method of claim 33, wherein said extraction medium is a solution comprising one or more acetate salts.

47. The method of claim 46, wherein said solution comprises ammonium acetate, sodium acetate, potassium acetate, magnesium acetate, calcium acetate, or a mixture thereof.

48. The method of claim 46, wherein said solution comprises ammonium acetate, sodium acetate, or a mixture thereof.

49. The method of claim 46, wherein said solution has a concentration of about 0.05 M to about 1 M with respect to acetate.

50. The method of claim 46 wherein said solution has a concentration of about 0.1 M to about 0.75 M with respect to acetate.

51. The method of claim 46, wherein said solution has a concentration of about 0.1 M to about 0.3 M with respect to acetate.

52. The method of claim 33, wherein said solution has a pH between about 5 and about 10.

53. The method of claim 33, wherein said solution has a pH between about 6 and about 9.2.

54. The method of claim 33, wherein said solution has a pH between about 6.2 and about 8.5.

55. The method of claim 33 wherein said pharmaceutical composition is provided as at least one unit dosage form selected from tablet, capsule, gel cap, buccal form, troche, and lozenge.

56. The method of claim 33, wherein said pharmaceutical composition is provided as a tablet form comprising a core and an outer layer, wherein said core comprises conjugated estrogens and said outer layer comprises bazedoxifene acetate.

57. The method of claim 55, further comprising removing any coating from said dosage unit prior to contacting the dosage unit with said extraction medium.

58. The method of claim 33, wherein the amount of said extraction medium used during the contacting of said pharmaceutical composition with said extraction medium to produce the suspension is from about 0.2 ml per dosage unit to about 10 ml per dosage unit.

59. The method of claim 33, wherein said pharmaceutical composition is contacted with said extraction medium for about 1 minute to about 120 minutes.

60. The method of claim 33, wherein said pharmaceutical composition is contacted with said extraction medium for about 1 minute to about 30 minutes.

61. The method of claim 33, wherein said pharmaceutical composition is contacted with said extraction medium for about 1 minute to about 5 minutes.

62. A method of separating a pharmaceutical composition comprising bazedoxifene acetate Form A and/or Form B and one or more components that produce X-Ray diffraction patterns having one or more interfering peaks at or near the characteristic peak or peaks for bazedoxifene acetate Form A and/or Form B, said method comprising:
(a) contacting said pharmaceutical composition with a solution comprising at least one acetate salt to produce a suspension, wherein bazedoxifene acetate Form A and/or Form B is substantially insoluble in the solution and wherein said one or more components are substantially soluble in the solution;
(b) centrifuging said suspension to produce a solid and a supernatant solution, wherein said one or more components are substantially contained in said supernatant solution; and
(c) collecting and drying the solid to produce a composition substantially free of said one or more components that produce X-Ray diffraction patterns having one or more interfering peaks.

63. The method of claim 62, wherein said pharmaceutical composition is provided as a tablet comprising a core and an outer layer, wherein said core comprises conjugated estrogens and said outer layer comprises bazedoxifene acetate Form A and/or Form B, and further comprising removing any coating from said tablet prior to contacting the tablet with said solution.

64. The method of claim 62, wherein said one or more components that produce X-Ray diffraction patterns having interfering peaks include lactose, sucrose, or a mixture thereof.

65. The method of claim 62, wherein the characteristic peak for bazedoxifene acetate Form A is at about 12.8±0.2° in 2theta angular degree by Cu radiation.

66. The method of claim 62, wherein the characteristic peaks for bazedoxifene acetate Form B are at about 12.0±0.2° and about 13.3±0.2° in 2theta angular degree by Cu radiation.

67. The method of claim 62, wherein said interfering peaks are at from about 11.9±0.2° to about 13.3±0.2° in 2theta angular degree by Cu radiation.

68. A method of detecting bazedoxifene acetate Form A and/or Form B in a pharmaceutical composition comprising bazedoxifene acetate Form A and/or Form B and one or more components that produce X-Ray diffraction patterns having one or more interfering peaks at or near the characteristic peak or peaks for bazedoxifene acetate Form A and/or Form B, said method comprising:
(a) producing a composition containing bazedoxifene acetate Form A and/or Form B by the method of any one of claims 33-67, wherein the composition is substantially free of said one or more components that produce X-Ray diffraction patterns having one or more interfering peaks;
(b) forming the composition containing bazedoxifene acetate Form A and/or Form B into a tablet or pellet for X-Ray diffraction measurement; and
(c) analyzing said tablet or pellet using X-Ray diffraction.
